(19) United States
(12) Patent Application Publication
(10) Pub. No.: US 2009/0164012 A1
(43) Pub. Date: Jun. 25, 2009

(54) Medical implant component and method for fabricating same

(75) Inventors: Haitong Zeng, Oakland, NJ (US); Zongtao Zhang, Unionville, CT (US); Balaji Prabhu, Allendale, NJ (US); Daniel E. Lawrynowicz, Cornwall, NY (US)

Correspondence Address: LERNER, DAVID, LITTENBERG,;KRUMHOLZ & MENTLIK, 600 SOUTH AVENUE WEST, WESTFIELD, NJ, 07090, US

(73) Assignee: Howmedica Osteonics Corp., Mahwah, NJ (US)

(21) Appl. No.: 12/005,138

(22) Filed: Dec. 21, 2007

(51) Int. Cl. A61F 2/02

(52) U.S. Cl. 623/11.11

(57) ABSTRACT

A medical implant component and a method for fabricating the same are provided. The medical implant component may comprise a substrate having a bearing portion, in which at least the bearing portion has a coating of at least 25 micrometers of a predetermined material. The predetermined material of the coating may include chromium ceramic having a chromium component which releases less than approximately 7 parts of hexavalent chromium per billion parts of water solution when the medical implant component is immersed in approximately 500 milliliters of water for approximately one week at a temperature in a range of room temperature to just below a boiling point of the water at atmospheric pressure. The residual stress of the coating may be less than approximately 100,000 pounds per square inch. The chromium ceramic may be a chromium oxide, a chromium carbide, a chromium nitride, or a chromium boride, or any combination thereof.

### CROSS-REFERENCE TO RELATED APPLICATIONS

This application is related to commonly owned concurrently filed U.S. patent application Ser. No. ______ (Attorney Docket No.: (OSTEONICS 3.0-686) entitled “SURFACE TREATMENT OF IMPLANTS” and commonly owned concurrently filed U.S. patent application Ser. No. ______ (Attorney Docket No.: OSTEONICS 3.0-685), entitled “CHROMIUM OXIDE POWDER HAVING A REDUCED LEVEL OF HEXAVALENT CHROMIUM AND A METHOD OF MAKING THE POWDER” the disclosures of which are hereby incorporated by reference herein.

### BACKGROUND OF THE INVENTION

1. Field of the Invention

The present invention relates to a medical implant component and a method for fabricating the same and, more particularly, to such medical implant which is fabricated with a predetermined material such as a chromium ceramic based material which is processed so as to have little or no hexavalent chromium.

2. Description of the Related Art

A medical implant component or components may be used within a patient for replacement surgery such as hip replacement surgery or the like. Such medical implant components may include femoral head components and acetabular cup components. With such components, a ball or mating portion of the femoral head component is adapted to mate with a mating portion of the acetabular cup component.

After a medical implant component is surgically implanted in a patient, a mating portion thereof (such as the ball portion of a femoral head component) will move many times within a mating portion of another medical implant component (such as that of the acetabular cup component) or, if a single medical implant component is used and affixed to a bone or the like of a patient, within or relative to such portion of the patient.

As is to be appreciated, the medical implant component or components should provide excellent wear capability so as to survive for a relatively long period of time. In an attempt to provide for such wear capability, one or both of the components may be coated with a predetermined material or materials. For example, the ball portion of the femoral head component may be coated with a predetermined coating material. Although such material may provide a fairly hard surface, scratches may still occur during normal use. Additionally, the coating may be applied by a chemical vapor deposition (CVD) process or a physical vapor deposition (PVD) process. These coating processes may enable only a relatively thin coating to be applied.

The use of a relatively thin coating on a mating or bearing surface of a medical implant component may result in a failure of the coating during actually use. As an example, consider the situation if a foreign material were to get into the joint between the ball portion of a femoral head component and the mating or bearing portion of an acetabular cup component. During movement, the foreign material may rub against the coating on the ball portion. As a result, a scratch or crack in the coating may develop which may spread into a larger crack. Additionally, other scratches or cracks may also develop and grow into larger cracks. Eventually, such crack or cracks may result in particles of the coating material being removed from or flaking off from such coating material. As is to be appreciated, such particles or flakes of the coating material inside a patient are not desirable.

Accordingly, the coatings on medical implant components may result in problems due to scratching. As a result, the wear life of the medical implant component or components may be adversely affected.

As such, it would be advantageous to provide a medical implant component which reduces the likelihood of scratches which may result in particles being removed therefrom. As a result, such medical implant component may have an excellent wear capability so as to increase the life of such component(s) in the patient.

### SUMMARY OF THE INVENTION

In accordance with an aspect of the present invention, a medical implant component is provided which comprises a substrate having a bearing portion, in which at least the bearing portion has a coating of at least 25 micrometers of a predetermined material. The bearing portion with the coating may be operable to articulate with a portion of another medical implant component or a portion of a patient. The predetermined material of the coating may include a chromium ceramic having a chromium component which releases less than approximately 7 parts of hexavalent chromium per billion parts of water solution when the medical implant component is immersed in approximately 500 milliliters of water for approximately one week at a temperature in a range of room temperature (approximately 20 degrees Centigrade) to just below a boiling point of the water (approximately 99 degrees Centigrade) at atmospheric pressure (1 Atm.). Additionally, the residual stress of the coating may be less than approximately 100,000 pounds per square inch.

In accordance with another aspect of the present invention, a medical implant component is provided which may be operable to articulate with a portion of another medical implant component or a portion of a patient. Such medical implant component may be formed of a predetermined material which may include a chromium ceramic having a chromium component which releases less than approximately 7 parts of hexavalent chromium per billion parts of water solution when the medical implant component itself is immersed in approximately 500 milliliters of water for approximately one week at a temperature in a range of room temperature (approximately 20 degrees Centigrade) to just below a boiling point of the water (approximately 99 degrees Centigrade) at atmospheric pressure (1 Atm.). Additionally, the residual stress of the predetermined material may be less than approximately 100,000 pounds per square inch.

In accordance with yet another aspect of the present invention, a method for fabricating a medical implant component is provided. Such method may comprise producing a substrate having a coating of at least 25 micrometers of a predetermined material applied to at least a bearing portion thereof, in which the predetermined material includes a chromium component, and performing a process to reduce hexavalent chromium from the predetermined material of the coating such that no more than approximately 7 parts of the hexavalent chromium is released per billion parts of water solution when the medical implant component is immersed in approximately 500 milliliters of water for approximately one week at a temperature in a range of room temperature (approximately 20 degrees Centigrade) to just below a boiling point of the water (approximately 99 degrees Centigrade) at atmospheric pressure (1 Atm.). The bearing portion with the coating may be operable to articulate with a portion of another medical implant component or a portion of a patient.

In accordance with still another aspect of the present invention, a method for fabricating a medical implant component which may be operable to articulate with a portion of another medical implant component or a portion of a patient is provided. Such method may comprise forming the medical implant component from a predetermined material, in which the predetermined material may include a chromium ceramic having a chromium component, and performing a process to eliminate hexavalent chromium from the chromium ceramic such that no more than approximately 7 parts of the hexavalent chrome is released in one billion parts of water solution when the medical implant component is immersed in approximately 500 milliliters of water for approximately one week at a temperature in a range of room temperature (approximately 20 degrees Centigrade) to just below a boiling point of the water (approximately 99 degrees Centigrade) at atmospheric pressure (1 Atm.).

### BRIEF DESCRIPTION OF THE DRAWINGS

A more complete appreciation of the subject matter of the present invention and the various advantages thereof can be realized by reference to the following detailed description in which reference is made to the accompanying drawings wherein like reference numbers or characters refer to similar elements.

FIG. 1 is a diagram of two medical implant components which are adapted to mate together;

FIG. 2 is a diagram of a medical implant component in accordance with an embodiment of the present invention;

FIG. 3 is a diagram to which reference will be made in explaining a method to reduce hexavalent chromium of a medical implant component in accordance with an embodiment of the present invention;

FIGS. 4A, 4B, 4C, and 4D are diagrams of temperature profiles to which reference will be made in explaining a process to reduce hexavalent chromium.

### DETAILED DESCRIPTION

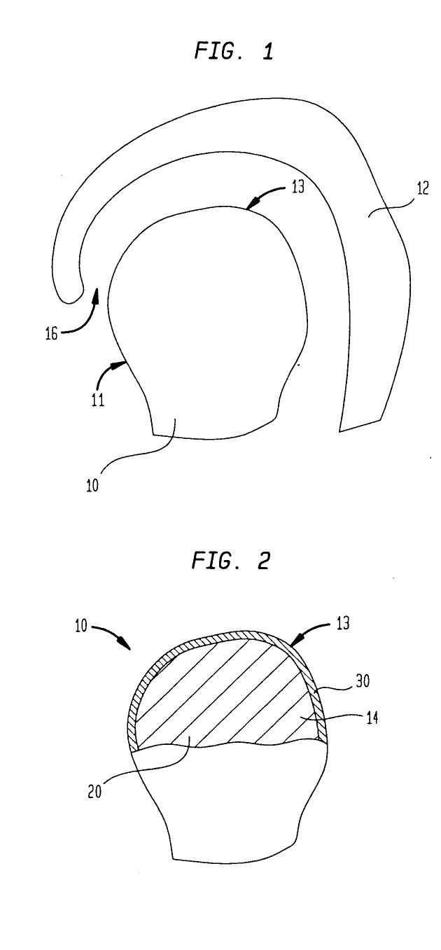

The present invention may be applied to a medical implant component and, in particular, to such component having a so-called bearing surface. As an example, reference is made to FIG. 1 which illustrates a femoral head 10 and an acetabular cup 12 which may be used in hip replacement surgery. Such femoral head 10 may be adapted to be inserted into the acetabular cup 12 when surgically placed within a patient. More particularly, during such placement, a bearing surface 13 of a ball portion 11 of the femoral head 10 may be inserted into a mating or insert portion 16 of the acetabular cup 12. To provide an acceptable mating condition, the bearing surface 13 may have a coating material applied thereto, as herein below more fully described.

FIG. 2 illustrates a partial cross-section of a medical implant component, such as the femoral head 10, in accordance with an aspect of the present invention. As shown therein, such component may include a coating 30 which has been applied to the outer surface of a substrate 20 of the femoral head 10. Such coating 30 may be applied to the entire outer surface of the substrate 20 or, alternatively, may be applied to only a predetermined portion thereof, such as the portion of the femoral head 10 which is to be inserted into the acetabular cup 12.

The material used for the coating 30 may have a chromium component such as chromium ceramic which may account for approximately 5-100% by weight or by volume of the total thereof. The chromium ceramic may be chromium oxide (such as Cr₃O₂ or CrO), chromium carbide (such as Cr₃C₂, Cr₇C₃, or Cr₂₃C₆), chromium nitride (CrN), or chromium boride (CrB), or any combination thereof. That is, for the coating 30, chromium ceramic may be used in the form of single compound (such as Cr₂O₃) or in the form of a multiple compound (such as Cr₂O₃, Cr₃C₂, and Cr₂₃C₆) . The chromium ceramic can be used as a primary phase of the coating 30, such as 100% chromium ceramic or 51-99% chromium ceramic. Additionally, the chromium ceramic can be used as a secondary phase of the coating 30, such as in a material having approximately 2-50% chromium ceramic such as 20% Cr₂O₃ dispersed in 80% Al₂O₃ or 10% CrN dispersed in 90% TiN. Additionally, such material for the coating 30 may be a biocompatible material such as a cermet having a carbide content of a predetermined amount such as at least 6.17 percent by weight such as that described in co-pending U.S. application entitled “Method for Fabricating a Medical Component from a Material Having a High Carbide Phase and Such Medical Component” with inventors Daniel E. Lawrynowicz, Aiguo Wang, and Zongtao Zhang and having U.S. application Ser. No. 11/728,676, filed Mar. 26, 2007, which is hereby incorporated by reference. Also see copending U.S. application entitled “Method for Fabricating a Biocompatible Material Having a High Carbide Phase and Such Material” with inventors Daniel E. Lawrynowicz, Aiguo Wang, Zongtao Zhang, and Haitong Zeng and having U.S. application Ser. No. 11/728,678, filed Mar. 26, 2007, which is hereby incorporated by reference. The coating material may have a composition of cermet that has a CoCrMo alloy as a matrix and multiple chromium ceramics dispersed in the metallic alloy such as chromium carbide, chromium oxide, and chromium nitride.

Chromium ceramic based materials are more chemically stable than metallic substrates such as cobalt chromium (CoCrMo), titanium alloys, stainless steel (316 L) and Zircornium alloys. It should be noted that the more chemically stable a material is, the less it may corrode in body fluid. Additionally, when used as the material for the coating 30, chromium ceramic based materials may provide a relatively hard surface. As an example, a coating formed from chromium oxide or chromium carbide may have a hardness value of approximately 800-2400 Vickers; whereas, a bearing surface of cobalt chromium may have a hardness value of only approximately 400 Vickers.

By utilizing a material such as chromium oxide or chromium carbide for an outer coating (such as coating 30) of a medical component (such as femoral head 10), may ensure that such medical component has a relatively hard surface and, as a result, is resistant to scratches. As is to be appreciated, such resistance to scratches may increase the operational life thereof and may prevent fragments or debris of the medical component from being released inside the patient which may be undesired and possibly harmful.

The coating 30 may be applied to the bearing portion 14 by a spraying process. Such spraying process may be a thermal type spraying process, such as a plasma spraying process or a high velocity oxygen fuel (HVOF) spraying process. The HVOF spraying process may be a gas fuel process such as a propane type process or, alternatively, may be a liquid fuel process such as a kerosene type process. Additionally, such spraying process may be performed by a so-called high velocity cold spraying process such as that described in co-pending U.S. application entitled “High Velocity Spray Technique for Medical Implant Components” with inventors Daniel E. Lawrynowicz, Aiguo Wang, and Eric Jones and having U.S. application Ser. No. 11/325,790, filed Jan. 5, 2006, which is hereby incorporated by reference.

The spraying process may be controlled or regulated such that a predetermined amount of coating material is applied to the substrate during a predetermined time interval or during each pass. More specifically, the spraying operation may be performed in an apparatus having a fixture for holding the medical implant component and a spray gun or nozzle from which the coating or spray material is supplied. During the spraying operation, either or both of the spray gun and/or fixture may move in a predetermined or controlled manner. For example, the fixture having the medical implant component may rotate at a predetermined rate in front of the spray gun. As a result, the amount of coating material which is applied to the substrate of the medical implant component during each revolution or pass may be controlled to a predetermined value. For example, such control may result in a thickness of coating material of approximately 10 to 12.5 microns or less being applied in each pass. Additionally, the spraying process may be controlled or regulated such that the coating material has a predetermined total thickness, such as 25 micrometers or more.

A thermal spray process is usually conducted in air. During such spray process, the chromium ceramic particles may be melted by a hot plasma flame (which may have a temperature greater than approximately 5000° C.) or by a high velocity flame (which may have a temperature greater than approximately 2000° C.), and then deposited on a metallic substrate. The small amount of melted particles may react with oxygen in the air at the high temperature, and form a gas phase hexavalent chromium compound, CrO₃ (g). Since the temperature of the metallic substrate may be low (such as between 100-250° C.), the CrO₃ (g) may be condensed into solid CrO₃(s) particles. The condensed CrO₃(s) may be trapped between the splats of multilayers of chromium ceramics coating. The inventors have found that coatings containing chromium ceramics contain a small amount of CrO₃(s) even when a thermal spray method in air is utilized.

The chromium ceramic material in the coating 30 of the medical implant or femoral head 10 may give off or release hexavalent chromium (Cr+6) after being inserted inside a patient. Such release may be due to a reaction within the patient such as that set forth in equation 1 below:

CrO₃+H₂O=2H⁺+CrO₄²⁻ (1)

In any event, hexavalent chromium may be harmful to a patient if a relatively large amount thereof were to be released inside a patient.

A description will now be provided on a technique for obtaining the amount of chromium six or CrO₃ from a medical implant having an outer layer or coating of chromium ceramic.

Initially, it should be noted that the trace amount of CrO₃ in the coating of chromium ceramic based materials may not be tested by a weight loss method. That is, since the coating bond on the metallic substrate of the medical implant is normally strong, any destructive test may adversely affect the test results. Hence, a non-destructive test method called an extraction method may be utilized. The test procedure for such method is described below.

A Soxhlet extractor may be used for this test method and the medical implant may be placed inside. The Soxhlet extractor may be attached to a 1000 ml flask filled with 500 ml reagent-grade deionized (DI) water, and a condenser cooled with water may be attached to the extractor. Both the weight of the added water and the total weight of the water, the flask and the extractor may be monitored before, during and after the test.

The flask may be heated with a heating mantel to boil the distilled water. As a result, water vapor may recondense in the condenser and water may drop into the extractor and accumulate. Gas tubing may be inserted into the extractor through an opening in the condenser to introduce air regulated with a pressure regulator and controlled with a flowmeter into the extractor so that the water in the extractor is enriched with oxygen. The water level in the flask may be checked daily and maintained. The temperature of the water in the extractor may be measured periodically; the temperature variation within each extraction may be monitored and recorded. The temperature may vary between room temperature (approximately 20 degrees Centigrade) to just below the boiling point of the water (approximately 99 degrees Centigrade) at atmospheric pressure (1 Atm.).

The concentration of chromium six (Cr6+) may then be measured in the extracted solution by ion Chromatography (IC). The sampling and testing may be conducted daily until the chromium six (Cr6+) concentration does not change with time. The non-change of chromium six (Cr6+) concentration indicates that all (or substantially all) of CrO₃ is extracted from the implant and into the water. Generally, the content of Cr6+ may be unchanged after approximately 24 hours of extraction. The concentration of Cr6+ in the extracted solution (C1) and the weight of extracting solution (W) may be recorded. From these values the total chromium six content (C) may be expressed as in equation 2 below:

C═C₁W (2)

And the total CrO₃ content (C₂) may be expressed as in equation (3) below:

C₂=1.923C₁W (3)

In equation 3, 1.923 is a ratio of the atomic weight of CrO₃ to the atomic weight of Cr.

To eliminate or reduce the above-mentioned harmful effects due to hexavalent chromium which may be present in the medical implant component having coating 30 with chromium ceramic, such implant may be subjected to one or more predetermined processes to cause hexavalent chromium to be released prior to actual use so that when the medical implant component is surgically placed inside the patient no hexavalent chromium or only a relatively small amount of hexavalent chromium may be released inside the patient. Such predetermined processes or methods are herein below described. Additionally, methods are herein below described which may enable the coating to be applied so as to have an acceptable and relatively low residual stress level (or residual torque). As an example, such residual stress level may have a value less than approximately 100,000 pounds per square inch.

The first method is a vacuum or low pressure plasma thermal spray method. Note that the CrO₃ may come from the reaction of oxygen (O₂) to melted or evaporated Cr₂O₃ or Cr₃C₂ particles. However, by using vacuum or low pressure plasma thermal spray, the entire spray may be protected by an inert gas, such as Argon (Ar), Helium (He), or Nitrogen (N₂). Since neither oxygen nor air is involved, CrO₃ may not be formed during the coating process.

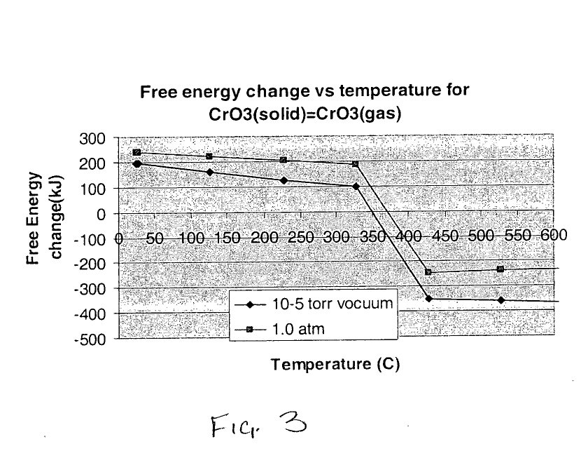

The second method is a heat treatment method which may be performed in a vacuum or flowing inert gas. If the coating is made by an air plasma or a high velocity oxygen fuel (HVOF) technique, low temperature heat-treatment may remove the CrO₃. Thermodynamically, and with reference to FIG. 3, CrO₃ may only be stable in a solid state at a temperature less than approximately 375° C. in ambient pressure and less than approximately 350° C. at 10⁻⁵ torr pressure. In other words, CrO₃ may decompose from a solid to a gas at a temperature greater than approximately 375° C. in ambient pressure and greater than approximately 350° C. at 10⁻⁵ torr pressure.

When the temperature becomes higher, the CrO₃ will vaporize into a gas. In addition to evaporation, CrO₃ may be decomposed into Cr₂O₃ and O₂. The actual process may be a combination of both evaporation and decomposition. The corresponding reactions may be expressed as follows:

CrO₃(s)=CrO₃ (g) (4)

2CrO₃(s)=Cr₂O₃ (s)+3/2O₂ (g) (5)

The melting point of CrO₃ is 196° C. (Handbook of Chemistry and Physics, 31 edition, 1949). When the temperature is over 196° C. (such as 200° C.), there may be a small balanced vapor phase of CrO₃. Theoretically, CrO₃ may be removed at a temperature over 196° C. when subjected to an inert gas flow for sufficient time. The flowing inert gas may blow the vapor phase of CrO₃ away, so that the rest of the liquidized CrO₃ is gradually removed. However, if the temperature increases to over the boiling point of CrO₃ (such as 350° C. at 10⁻⁵ torr pressure), the removal of the CrO₃ may be significantly increased, as described below.

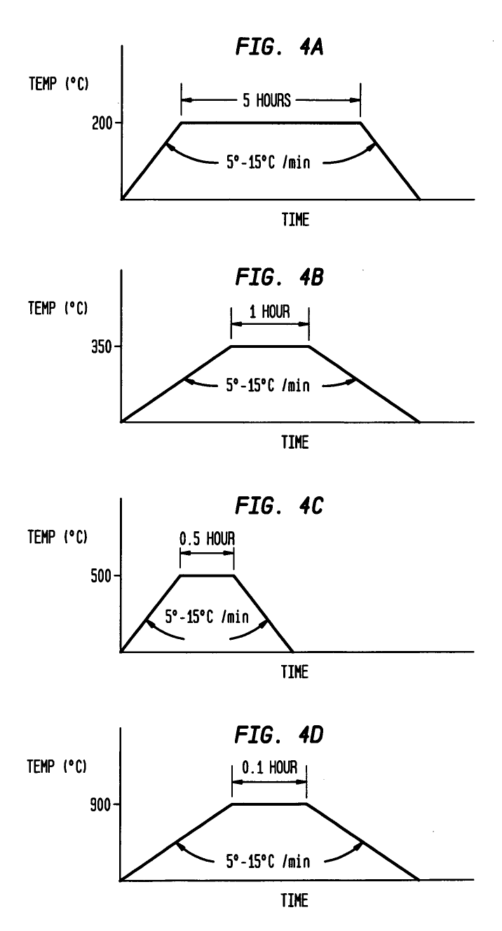

In the second method, the medical implant component having coating 30 with the above-described chromium ceramic (such as chromium oxide, chromium carbide, or a material, such as a chromium carbide based cermet material, having a carbide content of at least 6.17 percent by weight) may be placed into a furnace, such as a vacuum furnace, and subjected to a predetermined temperature/pressure profile. As a first example and with reference to FIG. 4A, the medical implant component may be placed inside the furnace and subjected to a low partial pressure such as by use of an inert gas. The temperature inside the furnace may be increased from room temperature at a predetermined rate such as between 5 and 15 degrees Centigrade per minute until the temperature reaches approximately 200° C. at which point the temperature may be maintained for a predetermined time such as approximately 5 hours. Thereafter, the temperature may be decreased at a predetermined rate such as between 5 and 15 degrees Centigrade per minute until the temperature reaches room temperature.

As a second example, the medical implant component may be placed inside the furnace at atmospheric pressure and subjected to the temperature profile shown in FIG. 4B. That is, the temperature inside the furnace may be increased from room temperature at a predetermined rate such as between 5 and 15 degrees Centigrade per minute until the temperature reaches approximately 350° C. at which point the temperature may be maintained for a predetermined time such as approximately 1 hour. Thereafter, the temperature may be decreased at a predetermined rate such as between 5 and 15 degrees Centigrade per minute until the temperature reaches room temperature.

If instead of 350° C. as in the second example the final temperature inside the furnace is increased, the dwell time thereat may be decreased. For example and with reference to FIG. 4C, the temperature inside the furnace may be increased from room temperature at a predetermined rate such as between 5 and 15 degrees Centigrade per minute until the temperature reaches approximately 500° C. at which point the temperature may be maintained for a predetermined time such as approximately 0.5 hour. Thereafter, the temperature may be decreased at a predetermined rate such as between 5 and 15 degrees Centigrade per minute until the temperature reaches room temperature. As another example and with reference to FIG. 4D, the temperature inside the furnace may be increased from room temperature at a predetermined rate such as between 5 and 15 degrees Centigrade per minute until the temperature reaches approximately 900° C. at which point the temperature may be maintained for a predetermined time such as approximately less than 0.1 hour. Thereafter, the temperature may be decreased at a predetermined rate such as between 5 and 15 degrees Centigrade per minute until the temperature reaches room temperature.

The third method is a washing technique which may utilize an acidic solution containing a reduction compound. Hexavalent chromium is a strong oxidizing agent, which can be reduced by a reducing agent in acidic solution (i.e., a solution having a pH<7). An example of a suitable acid and reduction reaction is provided below:

Cr₂O₃+2H₂CrO₄+H₂C₂O₄+3H₂SO₄═Cr₂O₃+Cr₂(SO₄)₃+2CO₂+6H₂O+O₂ (6)

wherein the chromium oxide (Cr₂O₃) coated implant is soaked in a mixture of water (H₂O), oxalic acid (H₂C₂O₄), and sulfuric acid (H₂SO₄). The sulfuric acid provides the acidic environment for the reaction to occur. Hexavalent chromium (Cr6+) in the chromium oxide (Cr₂O₃) reacts with the water to form chromic acid (H₂CrO₄). The chromic acid is reduced by the oxalic acid into trivalent chromium (Cr3+). The trivalent chromium reacts with the sulfuric acid to form chromium sulfate (Cr₂(SO₄)₃). The chromium sulfate is water soluble and non-toxic. The chromium sulfate is simply washed away with water. The carbon dioxide (CO₂) gas simply bubbles out of the solution. Because the solution is kept in a reducing condition, the concentration of the reducing agent is far higher than the dissolved oxidizing agent (O₂), there may not co-exist Cr⁶⁺ to Cr³⁺. This reaction reduces the hexavalent chromium present in the chromium oxide coating to a significantly reduced level. Experimental results indicate that less than approximately 7 parts of hexavalent chromium may be released per billion parts of water solution when the medical implant component is immersed in approximately 500 milliliters of water for approximately one week at a temperature in a range of room temperature (approximately 20 degrees Centigrade) to just below a boiling point of the water (approximately 99 degrees Centigrade) at atmospheric pressure (1 Atm.). As such, the hexavalent chromium may be reduced to a biocompatible level suitable for use on medial implants.

The acid reduction process is economical and easy to perform by simply adding a small amount of a reducing agent, such as 1-5%/wt Oxalic acid (H₂C₂O₄) into the acid washing solution. However, other reducing and water soluble compounds may be used in this step to remove the hexavalent chromium. For example, compounds containing Ni2+, Fe2+, Zn2+, Sn2+, etc. ions, sulfite acid (H₂SO₃, NaHSO₃) may alternatively be used. Oxalic acid and sulfite acid may be preferred because they do not introduce other metallic impurities into the washing process.

With regard to the above-described three methods, the vacuum spray is more expensive than air spray, while the liquid washing method may take a longer time. The air thermal spray plus post heat treatment in vacuum or inert gas environment may be the most economical method. However, the post heat-treatment may cause an adverse residual stress effect. Several methods may be utilized to control and minimize such residual stress. Such methods are herein below described.

The first method is to control the absolute value of residual stress. The inventors have found that the residual stress should be less than approximately 400 ksi to avoid spalling (i.e., a condition in which all or a part of the coating comes off) and cracking of chromium ceramic coating from the metallic substrate. However, a preferred residual stress value may be less than approximately 200 ksi, a more preferred residual stress value may be less than approximately 100 ksi, and the most preferred residual stress value may be less than approximately 50 ksi.

The second method is to control the coating thickness. The preferred chromium ceramic coating thickness may be less than approximately 1000 μm. Such coating thickness may be suitable so as to avoid or not cause cracking for selected coating and substrate combinations, but may not be suitable for all combinations. A more preferred coating thickness may be less than approximately 500 μm, which may avoid cracking for a majority of coating/substrate combinations. A still more preferred coating thickness may be less than approximately 400 μm, which may avoid cracking for a majority of combinations of chromium ceramic coatings on metallic substrates. However, the most preferred coating thickness may be less than approximately 200 μm. Such thickness may avoid cracking for all or substantially all combinations of chromium ceramic coatings on a metallic substrate and may result in a relatively low residual stress after post heat treatment.

The third method is to control the heat-treatment temperature. The threshed temperature of the post heat-treatment may be approximately 900° C. When the temperature is higher than 900° C., the residual stress may be over 400 ksi. In such situation, the coating may be readily separated from the substrate. The preferred heat-treatment temperature may be lower than 700° C., whereupon the residual stress may be lower than approximately 200 ksi. The more preferred heat-treatment temperature may be less than 500° C., whereupon the residual stress may be lower than approximately 100 ksi. The most preferred heat-treatment temperature may be between 200-400° C., whereupon the residual stress may be lower than approximately 50 ksi.

The fourth method is to control the dwell time of heat-treatment. The preferred dwell time may be less than 10 hours. The more preferred dwell time may be less than 5 hours and the most preferred dwell time may be less than 1 hour.

The above third and fourth methods may be related. That is, thermodynamically, the metallic substrate and coating material may have interfacial reactions during the heat treatment which, as an example, may be expressed as follows:

2Ti+Cr₂O₃₋ₓ═Ti₂O₃₋ₓ+2Cr (7)

3Ti+2Cr₂O₃═3TiO₂+4Cr (8)

Volume shrinkage may occur for reactions (7) and (8), resulting in compressive stress in a tangential direction and tensile stress in a radial direction of the medical implant, such as a femoral head. This interfacial chemical reaction may cause the interfacial layer to become thicker at a high temperature and a long dwell time. The inventors have found that such interfacial layer was formed at a temperature above 700° C. and a dwell time of 10 hours. Therefore, the temperature and dwell time should be selected so as to balance complete or substantially complete removal of chromium six and keep residual stress as low as possible. At a lower temperature range (such as 200-500° C.), the dwell time may be relative long such as 1-5 hours. At a higher treatment temperature (such as 500-900° C.), the dwell time may be shorter (e.g., less than approximately 1.0 hour). In any event, the temperature and dwell time should be such that less than approximately 7 parts of chromium six or hexavalent chromium per billion parts of water solution is released when the medical implant component is immersed in approximately 500 milliliters of water for approximately one week at a temperature in a range of room temperature (approximately 20 degrees Centigrade) to just below a boiling point of the water (approximately 99 degrees Centigrade) at atmospheric pressure (1 Atm.) and the residual stress is less than approximately 100 ksi.

The fifth method is to select a combination of coating materials and substrate material(s), so that the thermal expansion coefficient mismatch therebetween may be less than approximately 10×10⁻⁶/° C. As an example thereof, Cr₂O₃, Cr₃C₂, or CrN may be used as a coating material on a CoCrMo substrate. A more preferred thermal expansion coefficient mismatch may be less than approximately 5×10⁻⁶/° C. As an example thereof, 50% Cr₂₃C₆ and 50% CoCrMo may be used for the coating on a CoCrMo substrate. The most preferred thermal expansion coefficient mismatch may be less than approximately 2×10⁻⁶/° C. As an example thereof, Cr₂O₃, Cr₃C₂, Cr₇C₃, or Cr₂₃C₆ may be used as the coating material on a Ti6Al4V substrate.

The sixth method is to consider the geometry of implant. As an example, a small size radius may cause a high residual stress to occur. As such, a small implant may have a higher residual stress concentration than a large implant. A preferred radius for an implant may be over 11 mm, a more preferred radius for an implant may be more than 16 mm, and the most preferred radius for an implant may be more than 20 mm. Thus, a large diameter in an implant is beneficial in that it may result in a low residual stress. Another factor of the geometry to consider is whether the implant has a convex or concave profile. As an example, with regard to the coating on a convex metallic surface such as a femoral head implant, the thermal expansion coefficient of the coating should be smaller than that of the substrate. An example thereof is Cr₂O₃ for the coating and Ti6Al4V for the ball well. As another example, with regard to the coating on a concave metallic surface such as a femoral cup implant, the thermal expansion coefficient of the coating should be larger than that of the substrate. An example thereof is 65% Cr₃C₂ and 35% CoCr for the coating on a Ti6Al4V cup. Accordingly, by considering the geometry of the implant, residual stresses may be reduced or minimized as compared to other designs.

Although in the above description the implant may have been formed of a chromium ceramic coating on a metallic substrate, the present invention is not limited thereto. Further, it should be noted that the same principles discussed above pertaining to a Cr6+ impurity level and residual stress control may be applied to an implant made of bulk chromium ceramics with the same composition of the coating. Thus, the present invention may be applied to an implant and/or coating formed of different materials.

Several detailed examples will now be described.

### EXAMPLE 1

Vacuum or low pressure plasma spray Cr₂O₃: A 41.8 mm diameter Ti6Al4V femoral head was sandblasted at 70 PSI using #30 Al₂O₃ sand. After ultrasonic cleaning in acetone, the roughed surface head was placed in a vacuum chamber. A mechanical pump was used to reduce the pressure inside the chamber to 10⁻³ torr. The pump was stopped and 99.99% Ar was fed into the chamber until the pressure was 1.0 atmosphere. This process was repeated three times and the chamber was kept at 10⁻³ torr. A SG-100 plasma gun was equipped inside the vacuum chamber. A robot carried the plasma gun to spray Cr₂O₃ powder on the sand-blasted ball. The plasma spray was conducted at 800 A, 38 Volts, Ar was the primary gas and He was the secondary gas, 5.0 inch spray distance, preheat temperature 500° F., and feed rate 2.0 lbs/hour. After the thermal spray, the ball head was mechanically ground and polished to a coating thickness of approximately 100 μm and the finished ball had a diameter of 42.00 mm. The finished ball was tested by a traction method for Cr6+ release from the coating to the traction solution using an ion chromatography (IC) method. The total soluble chromium was tested by inductively coupled plasma (ICP). The IC test showed that the total soluble chromium content was equal to the Cr6+ content. The Cr6+ release after 1 day and 1 week traction was non-detectable with a 5 part per billion (ppb) test limit. The residual stress on the surface of the as-sprayed coating was compressive at a range of 20 to 30 ksi in a tangential direction.

### EXAMPLE 2

Vacuum or low pressure plasma spray Cr₃C₂: all the procedures were the same as in example 1, except that the coating is Cr₃C₂. The Cr6+ release after 1 day and 1 week traction was non-detectable (i.e., less than the 5 ppb test limit). The residual stress on the surface of the as-sprayed coating was compressive at a range of 44 to 57 ksi in a tangential direction.

### EXAMPLE 3

Vacuum or low pressure plasma spray cermet: all the procedures were the same as in example 1, except that the coating was 75 wt % Cr₂₃C₆+25% F75 CoCrMo alloy on F75 CoCr alloy. The Cr6+ release after 1 day and 1 week traction was non-detectable (i.e., less than the 5 ppb test limit). The residual stress on the surface of the as-sprayed coating was compressive at 3 ksi to tensile at 25 ksi in a tangential direction.

### EXAMPLE 4

Vacuum or low pressure heat treatment of air plasma sprayed Cr₂O₃: 41.8 mm diameter Ti6Al4V femoral heads were sandblasted at 70 PSI using #30 Al₂O₃ sand. After ultrasonic cleaning in acetone, the roughed surface head was coated with a Cr₂O₃ coating. The coating conditions were SG-100 plasma gun, 900 A, 38 Volts, Ar was the primary gas and He was the secondary gas, 5.0 inch spray distance, preheat temperature 200° F., and feed rate 3.0 lbs/hour.

After thermal spray, the ball head was mechanically ground and polished to a coating thickness of approximately 200 μm for heat treatment and to test Cr6+ ions release and total soluble chromium afterwards. The pressure in the furnace was reduced to 10⁻⁵ torr and increased back up two times, and then kept at an argon pressure of 300 torr. Before heat treatment, a thermodynamic calculation was conducted to determine the heat treatment temperature. As indicated in FIG. 3, the minimum temperature to remove CrO₃ debris is approximately 350° C. at 10⁻⁵ torr pressure and 375° C. at 760 torr pressure.

As indicated above the coating thickness for the implant in example 1 above was 100 micrometers and that of example 4 was 200 micrometers. Additionally, the coating thickness for the implant in example 2 above was 300 micrometers, and that for the implant in example 3 above was 100 micrometers.

With reference to Table 1, it can be seen that without any heat treatment, the as-sprayed Cr₂O₃ coating contained 86 to 96 ppb Cr6+, which correspond to 0.0795 to 0.0928 total soluble chromium (i.e., almost all soluble chromium in the coating existed in Cr6+ form). After heat treatment at or above 350° C., both Cr6+ and total soluble chromium were non-detectable at 7 days traction period (i.e., less than the 5 ppb test limit). However, as the temperature increased, the residual stress also increased.

TABLE 1
Experimental results of Cr₂O₃ coating heat-treated at
different temperatures in a vacuum.

| | | Total | |
|---|---|---|---|
| | Cr6+, by | soluble | Residual |
| Temperature | IC | Cr, by ICP | stress, |
| and time | Ppb | ppm, | ksi |
| No heat | 81 to 96 | 0.0795 to | −28 to −32 |
| treatment | | 0.0928 | |
| 350° C. × 1 h | Non | Non | −28 to −32 |
| | detectable | detectable | |
| 350° C. × 10 h | Non | Non | −25 to −35 |
| | detectable | detectable | |
| 550° C. × 10 h | Non | Non | −34 to −49 |
| | detectable | detectable | |
| 750° C. × 10 h | Non | Non | −62 to −89 |
| | detectable | detectable | |
| 950° C. × 6 h | Non | Non | −188 to |
| | detectable | detectable | −199 |
| 1250° C. × 2 h | Non | Non | −360 to |
| | detectable | detectable | −410 |

Therefore, the present invention provides a medical implant component (and a method for fabricating the same) which may have excellent wear capability so as to provide a long life after being surgically implanted in a patient. Such medical implant component may be fabricated from a predetermined material or may have a coating on at least a portion thereof (such as a bearing portion) of a predetermined material. The predetermined material may include chromium ceramic having a chromium component. The medical implant component may be fabricated by or subjected to one or more predetermined processes so as to remove all or substantially all hexavalent chromium. That is, such predetermined processing may cause the medical implant component to have little or no hexavalent chromium afterwards. As an example, such processing may cause the medical implant component to release 7 parts or less, or even 5 parts or less, of hexavalent chromium per billion parts of water solution or water which may be measured when the medical implant component by itself is immersed in approximately 500 milliliters of water for approximately one week at a temperature in a range of room temperature to just below a boiling point of the water at atmospheric pressure.

The predetermined processes may include a vacuum or low pressure plasma thermal spray method, a heat treating method, and/or a washing method. Additionally, other methods or techniques may be utilized to remove all or substantially all of the hexavalent chromium. For example, a process may be utilized which involves placing the medical implant component into a predetermined liquid, such as water, and causing the water to be moved by ultrasonic agitation.

The coating may have a thickness of at least 25 micrometers of the predetermined material. Furthermore, the coating may be applied by one of a thermal spray process, a physical vapor deposition process, a chemical vapor deposition process, a cold spray process, an anodizing process, or an electroplating process.

Additionally, the medical implant component may be designed and/or fabricated so as to have a value of a residual stress of the coating less than a predetermined value. Such predetermined value may have a value of approximately 100,000 pounds per square inch.

Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the appended claims.

1. A medical implant component comprising a substrate having a bearing portion, in which at least said bearing portion has a coating of at least 25 micrometers of a predetermined material, said bearing portion with the coating being operable to articulate with a portion of another medical implant component or a portion of a patient, in which the predetermined material of the coating includes chromium ceramic having a chromium component which releases less than approximately 7 parts of hexavalent chromium per billion parts of water solution when the medical implant component by itself is immersed in approximately 500 milliliters of water for approximately one week at a temperature in a range of room temperature to just below a boiling point of the water at atmospheric pressure, and in which a value of a residual stress of the coating is less than approximately 100,000 pounds per square inch.

2. The medical implant component according to claim 1, in which an entire surface of the medical implant component is coated with the predetermined material.

3. The medical implant component according to claim 1, in which the chromium ceramic is a chromium oxide, a chromium carbide, a chromium nitride, or a chromium boride, or any combination thereof or in which the predetermined material is a material having a carbide content of at least 6.17 percent by weight.

4. The medical implant component according to claim 1, in which the chromium ceramic is approximately 5-100% by weight or volume of the predetermined material.

5. The medical implant component according to claim 1, in which the coating has a thickness value less than approximately 1000 micrometers.

6. A medical implant component operable to articulate with a portion of another medical implant component or a portion of a patient, said medical implant component being formed of a predetermined material, in which the predetermined material includes chromium ceramic having a chromium component which releases less than approximately 7 parts of hexavalent chromium per billion parts of water solution when the medical implant component itself is immersed in approximately 500 milliliters of water for approximately one week at a temperature in a range of room temperature to just below a boiling point of the water at atmospheric pressure and in which a value of a residual stress of the predetermined material is less than approximately 100,000 pounds per square inch.

7. The medical implant component according to claim 6, in which the chromium ceramic is a chromium oxide, a chromium carbide, a chromium nitride, or a chromium boride, or any combination thereof, or in which the predetermined material is a material having a carbide content of at least 6.17 percent by weight.

8. The medical implant component according to claim 6, in which the chromium ceramic is approximately 5-100% by weight or volume of the predetermined material.

9. A method for fabricating a medical implant component, said method comprising:
producing a substrate having a coating of at least 25 micrometers of a predetermined material applied to at least a bearing portion thereof, in which the predetermined material includes a chromium component; and
performing a process to reduce hexavalent chromium from the predetermined material of the coating such that no more than approximately 7 parts of hexavalent chromium is released per billion parts of water solution when the medical implant component is immersed in approximately 500 milliliters of water for approximately one week at a temperature in a range of room temperature to just below a boiling point of the water at atmospheric pressure,
said bearing portion with the coating being operable to articulate with a portion of another medical implant component or a portion of a patient.

10. The method according to claim 9, in which the predetermined material includes a chromium oxide, a chromium carbide, a chromium nitride, or a chromium boride, or any combination thereof, or the predetermined material is a material having a carbide content of at least 6.17 percent by weight.

11. The method according to claim 9, in which the chromium component is chromium ceramic and in which the chromium ceramic is approximately 5-100% by weight or volume of the predetermined material.

12. The method according to claim 9, in which an entire surface of the substrate is coated with the predetermined material.

13. The method according to claim 9, in which the coating has a thickness value less than approximately 1000 micrometers.

14. The method according to claim 9, in which a value of a residual stress of the coating is less than approximately 100,000 pounds per square inch.

15. The method according to claim 9, in which the process involves exposing the substrate having the coating to a predetermined temperature at a predetermined pressure for a predetermined time period.

16. The method according to claim 15, in which the predetermined temperature is approximately 550° C. or lower.

17. The method according to claim 16, in which the predetermined pressure is approximately atmospheric pressure or less.

18. The method according to claim 9, in which the process involves exposing the substrate having the coating to a temperature of approximately 900° C. or lower in a vacuum.

19. The method according to claim 9, in which the process involves causing the water to be moved by ultrasonic agitation.

20. The method according to claim 9, in which the coating is applied by one of a thermal spray process, a physical vapor deposition process, a chemical vapor deposition process, a cold spray process, an anodizing process, or an electroplating process.

21. A method for fabricating a medical implant component operable to articulate with a portion of another medical implant component or a portion of a patient, said method comprising: forming the medical implant component from a predetermined material, in which the predetermined material includes chromium ceramic having a chromium component; and performing a process to eliminate hexavalent chromium from the chromium ceramic such that no more than approximately 7 parts of the hexavalent chromium is released in one billion parts of water solution when the medical implant component is immersed in approximately 500 milliliters of water for approximately one week at a temperature in a range of room temperature to just below a boiling point of the water at atmospheric pressure.

22. The method according to claim 21, in which the chromium ceramic is a chromium oxide, a chromium carbide, a chromium nitride, or a chromium boride, or any combination thereof, or in which the predetermined material is a material having a carbide content of at least 6.17 percent by weight.

23. The method according to claim 21, in which the chromium ceramic is approximately. 5-100% by weight or volume of the predetermined material.

24. The method according to claim 21, in which the process involves exposing the medical implant component to a predetermined temperature at a predetermined pressure.

25. The method according to claim 24, in which the predetermined temperature is approximately 550° C. or lower.

26. The method according to claim 25, in which the predetermined pressure is approximately atmospheric pressure or less.

27. The method according to claim 21, in which the process involves exposing the medical implant component to a temperature of approximately 900° C. or lower in a vacuum.

28. The method according to claim 21, in which the water is a reducing water solution with a pH value less than 7 and in which the process involves causing the water solution to be moved by ultrasonic agitation.
